(19) United States
(12) Patent Application Publication
(10) Pub. No.: US 2015/0157378 A1
(43) Pub. Date: Jun. 11, 2015

(54) MATERIAL DELIVERY DEVICE

(71) Applicant: NLT SPINE LTD., Kfar Saba (IL)

(72) Inventors: Oded Loebl, Tel Mond (IL); Didier Toubia, Raanana (IL)

(73) Assignee: NLT SPINE LTD., Kfar SAba (IL)

(21) Appl. No.: 14/390,032

(22) PCT Filed: Apr. 4, 2013
(86) PCT No.: PCT/IB2013/052692

§ 371 Date: Oct. 2, 2014

Related U.S. Application Data
(60) Provisional application No. 61/620,467, filed on Apr. 5, 2012.

(51) Int. Cl. A61B 17/88

(52) U.S. Cl. CPC A61B 17/8822; A61B 17/8816

(57) ABSTRACT

Devices for introducing materials into the body include collapsible material-filled tubes with pressure members, movable along the tubes. When the pressure members are advanced toward the tube opening, the tube collapses causing material to be emitted from the tube to the desired location or surgical site in the body.

### CROSS-REFERENCES TO RELATED APPLICATIONS

This application is related to and claims priority from commonly owned U.S. Provisional Patent Application Ser. No. 61/620,467, entitled: Bone Material Delivery Device, filed on Apr. 5, 2012, the disclosure of which is incorporated by reference herein.

### TECHNICAL FIELD

The present invention is directed to devices for introducing materials into the body.

### BACKGROUND

Numerous surgical procedures, including spinal procedures such as spinal fusions for lumbar vertebrae, require surgeons introducing material into the body. In many instances, the materials cannot be delivered through conventional needles or other fine flow passages. Additionally, the materials may be delivered inaccurately, causing too little or too much material to be delivered to the surgical site. In the case of too much material, this material may migrate from the surgical site into the internal cavities of the body, causing adverse events. Moreover, the delivery systems that transport large (bulk) volumes of material are cumbersome, complicated and bulky.

### SUMMARY

The present invention provides devices, systems and methods for introducing materials into the body which cannot be delivered through needles and other fine now passages. The present invention allows these materials to be delivered accurately to the surgical site, and in precise amounts. The materials deliverable to the surgical site include particulates, such as powders, pastes, granules, puttys, viscous fluids, and mixtures thereof. For example, when the present invention is used in spinal fusion surgery, a fusion promoting material, natural or artificial, is delivered accurately through a small path or channel, and in the precise amount into the vertebral disk space or into an implant located in the disk space.

The delivery system of the present invention provides a guide member or delivery conduit for use with a delivery mechanism. The delivery mechanism includes a tube with a proximal and distal end. The tube is at least partially filled with material, such as bone fusion promoting material (FPM). A pressure member is positioned on the tube, typically at the proximal and slideable thereon, such that sliding the pressure member axially towards the distal end along the tube on the tube flattens or collapses the tube, causing material to be squeezed out of the tube, exiting the tube from the tip of the distal end, for delivery to the surgical site.

An embodiment of the invention is directed to a system for delivering material to a surgical site. The system comprises a collapsible tube (tube) and a pressure member movably mounted to the collapsible tube. The collapsible tube includes a proximal and a distal end, and is constructed for being filled at least partially with material. The pressure member is moveably mounted to the proximal end of the collapsible tube, and it is configured for moving distally along at least a majority of the collapsible tube, such that the distal movement of the pressure member collapses a portion of the collapsible tube and material is emitted from the distal end of the collapsible tube.

Another embodiment of the invention is directed to a method for delivering material to a surgical site. The method comprises providing a material delivery system. The material delivery system comprises a collapsible tube (tube) including a proximal and a distal end, the collapsible tube filled at least partially with material, and, a pressure member moveably mounted to the proximal end of the collapsible tube, the pressure member configured for moving distally (e.g., axially) along at least a majority of the collapsible tube, including, for example, along at least the majority of the initially-filled portion of the tube. The collapsible tube is such that the distal movement of the pressure member collapses a portion of the collapsible tube and material is emitted from the distal end of the collapsible tube. The surgical site is accessed with the material delivery system, such that the distal end of the collapsible tube is at a location least proximate to the surgical site, and, material is delivered to the surgical site by moving the pressure member distally along the collapsible tube, such that the tube collapses and the collapse causes material to be emitted from the collapsible tube at the distal end.

### BRIEF DESCRIPTION OF THE DRAWINGS

Attention is now directed to the drawing figures where like reference numerals or characters refer to corresponding or like components. The drawing figures are as follows.

FIG. 1 is a perspective view of an apparatus in accordance with the invention;

FIG. 2 is a perspective view of a tube used with the apparatus of the present invention;

FIG. 3 is a perspective view of another tube used with the apparatus of the present invention;

FIG. 4 shows material packaged in a tube used with the apparatus of the present invention;

FIGS. 5A and 5B show a plug of material in the tube used with the apparatus of the present invention;

FIG. 6 is a perspective view of another tube used with the apparatus of the present invention;

FIGS. 7A and 7B are perspective views of a portion of the apparatus of the present invention in an exemplary operation;

FIGS. 8A and 8B are perspective views of a tube and pressure member in accordance with the present invention;

FIG. 9A is a perspective view of an alternative pressure member used with the apparatus of the present invention;

FIG. 9B is a front view of the pressure member of FIG. 9A;

FIG. 10A is a perspective view of an alternative pressure member used with the apparatus of the present invention;

FIG. 10B is a front view of the pressure member of FIG. 10A;

FIG. 11 is a perspective view of an alternative pressure member used with the apparatus of the present invention;

FIGS. 12A and 13A are perspective views of tubes and alternative embodiments of pressure members;

FIGS. 12B and 13B are perspective views of tubes and alternative embodiments of the pressure members of FIGS. 12A and 13A, respectively;

FIGS. 12C-1 and 12C-2 are perspective views of alternative rollers for the pressure members of FIGS. 12A and 12B; and,

FIGS. 14A and 14B are perspective views of an exemplary operation in accordance with the present invention.

### DETAILED DESCRIPTION OF THE DRAWINGS

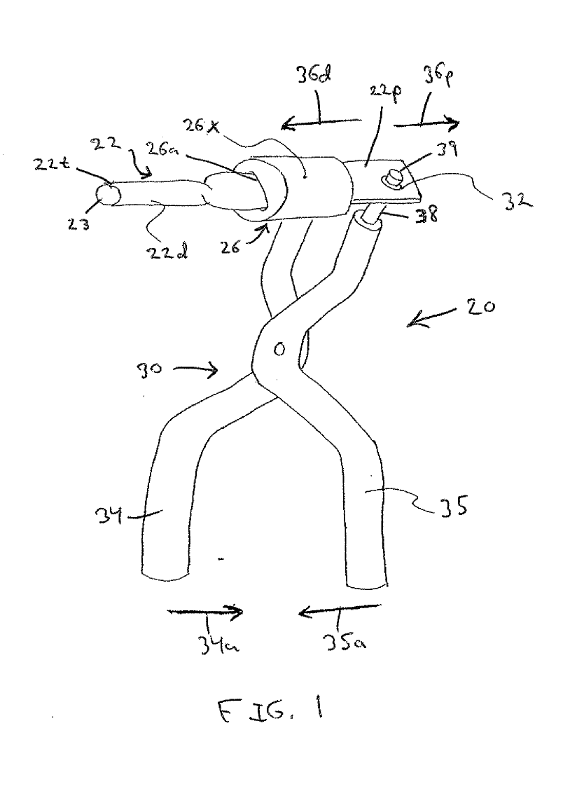

FIG. 1 shows an apparatus 20 in accordance with an embodiment of the invention. The apparatus 20 includes a tube 22, having a distal end 22d and a proximal end 22p, with a tip 22t at the distal end 22d, the tip 22t being open, so as to define an opening 23 in the tube 22. Throughout this document, the terms “distal” (including distally) and “proximal” (including proximally) are also descriptive of directions, with the distal direction being toward the tip 22t and opening 23, while the proximal direction is the direction away from the tip 22t and the opening 23.

A pressure member 26 is slideably connected to the tube 22 at the proximal end 22p. A handle 30 attaches to the pressure member 26, and the proximal end 22p of the tube 22 at an aperture 32. The handle 30 is formed, for example, of pivotally mounted arms 34, 35, which when gripped by an operator, move the pressure member 26 distally, in the direction of the arrow 36d (as arm 34 moves proximally, in the direction of the arrows 34a), and the proximal end 22p of the tube 22 proximally, in the direction of arrow 36p (as arm 35 moves distally, in the direction of the arrow 35a), collapsing the tube 22 and causing material to flow out of the tube via the opening 23. Arm 35 connects to the proximal end 22p of the tube 22 through an extension 38, which extends through the aperture 32 and a fixed to the proximal end 22p of the tube 22 by a connector 39.

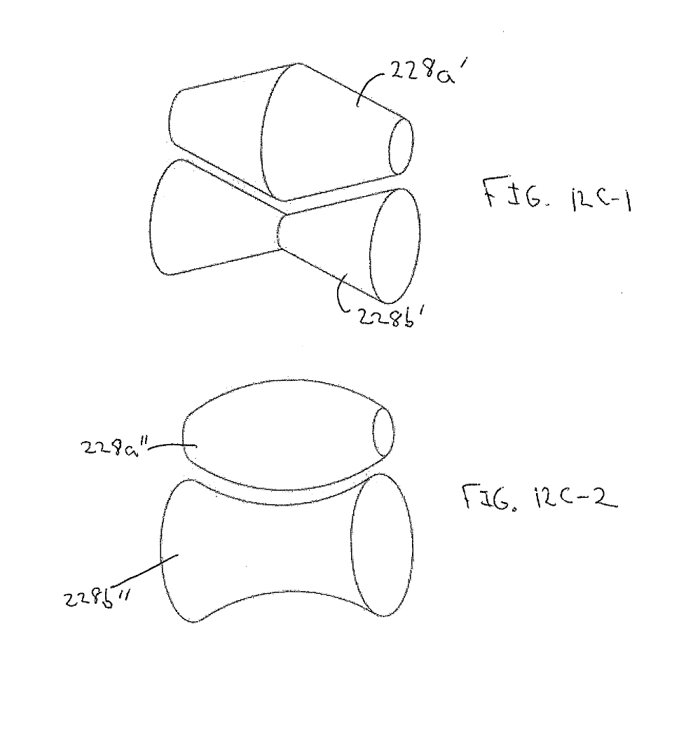

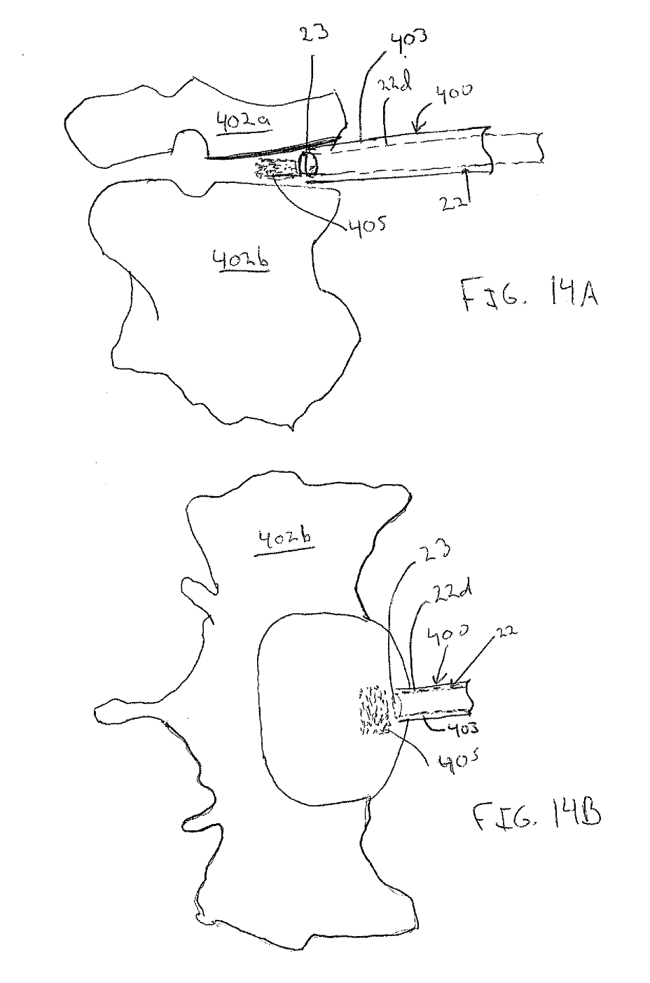

The tube 22 is, for example, cylindrical and of a rounded cross section. The tube 22 is, for example, made of a bendable or collapsible material such as plastic, metal, or the like, typically surgical grade, which is open at the distal end, i.e., the tip 22t. The material for the tube 22 may be plastically (permanent) or elastically (temporarily) deformable. The tube 22 may be made from single or multiple pieces. The tube 22 may include a hardened, rigid, or non-collapsible portion 22dx of the distal end 22d (in the region of the opening 23), that serves as a stop for the pressure members 226, 326, limiting the distal movement of the pressure members 226, 326, as shown in, for example, in FIGS. 12A, 12B, 13A and 13B.

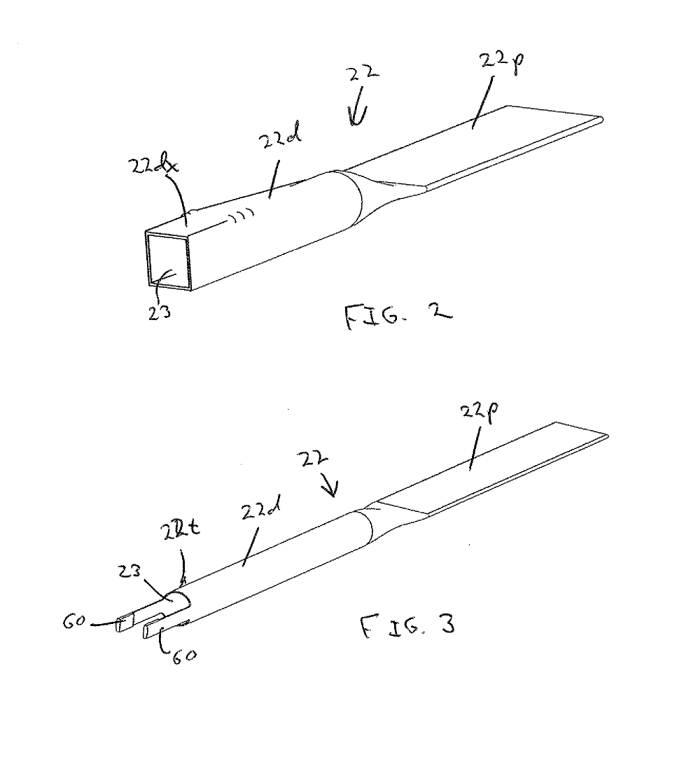

The tube 22 may also be of different cylindrical (or tubular) configurations, such as that shown in FIG. 2, where the distal end 22d is formed of a cylinder 22dx of rectangular, including square, cross section (while the remainder of the distal end 22d is circular, or rounded in cross-section). Additional tube 22 cross-sectional shapes include oval, elliptical, triangular or any other geometry. The tube 22 may include protrusions 60 which extend from the tip 22t at the distal end 22d, as shown in FIG. 3. These protrusions serve to attach or engage the surgical site, including interlocking with the surgical site, to allow for additional accuracy in the material delivery process to the surgical site.

The tube 22 may be filled with various materials for medical treatments. These materials may include, for example, fusion promoting materials, bone fill materials and bone cements and numerous other materials, both natural and artificial and combinations thereof. The materials may in various forms, for example, particulates, such as powders, pastes, granules, puttys, viscous fluids, and mixtures thereof. The materials may also be in solid form, in blocks 68, for example, as shown in FIG. 4, or semisolid form, so long as the material is of a viscosity that will allow it to be dispensed from the tube 22 upon the squeezing or collapsing of the tube 22. The materials may also include autogeneous bone graft, demineralized bone matrix, and the like.

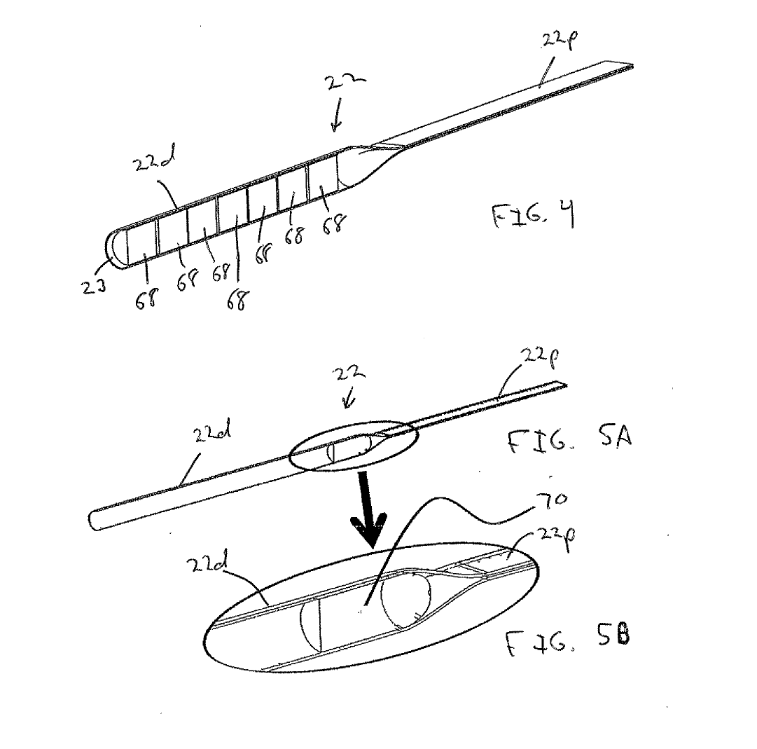

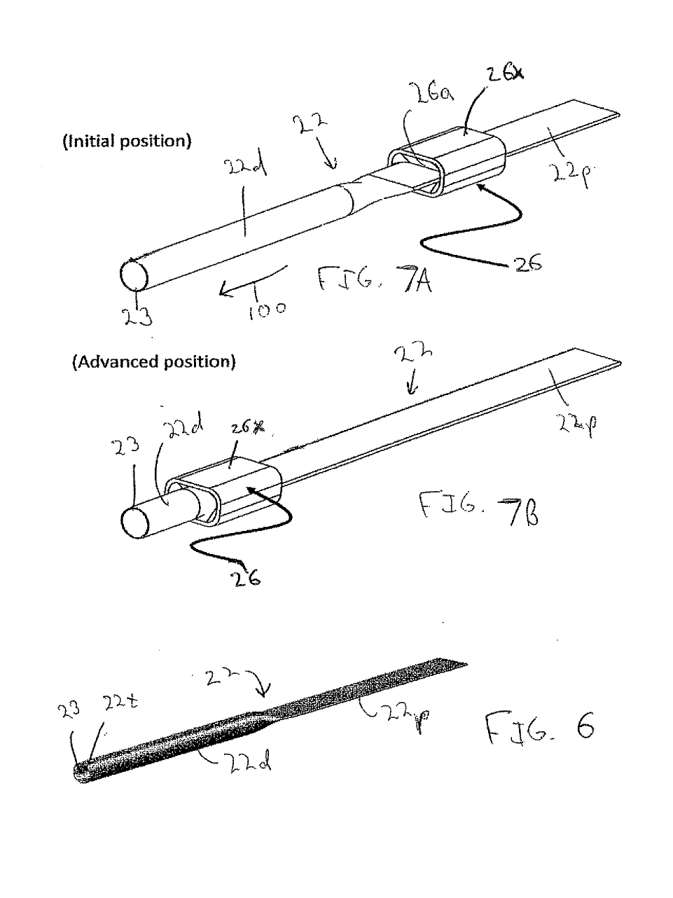

The tube 22 can also include a plug 70, positioned proximally in the distal end 22d of the tube 22. The plug 70 is pushed by the pressure member 26 (e.g., FIGS. 1, 7A and 7B) as it moves distally, as shown in FIGS. 5A and 5B. The plug 70 pushes the material (in the tube 22) distally, as the portion of the tube 22 at and behind (proximal to) the plug 70 is collapsed.

The tube 22 may be made from single or multiple pieces. For example, the tube 22, as shown in FIG. 6, is made of two parts, a first component, a cylinder of rounded cross section, for example, forming the distal end 22d, and a flattened portion, for example, forming the proximal end 22p. A sheet of material, as detailed above, is rolled into the cylindrical and flattened portions, and bonded along a seam, by conventional plastic and metal bonding techniques. The material for the sheet, for example, is a translucent material, such that the material may be viewed inside of the tube 22. The tube 22 may be prefilled with material upon manufacturing, or filled proximate to or at the time of use.

The pressure member 26 is configured for moving along the tube 22, distally, in order to collapse the tube 22, such that the pressure of the collapse causes material to be emitted from the tube 22 at the tip 22t through the opening 23. The pressure member 26 includes a block 26x, with a slot 26a or aperture extending therethrough, for receiving the tube 22, for example, at the proximal end 22p (and where applicable, the proximal end 22p which is flattened, as shown in FIG. 1) The slot 26a (or aperture) serves as a constrictor, forcing a reduction in the material-filled cross-sectional area of the tube 22, as the pressure member 26 is advanced axially (distally) toward the distal end 22d and the opening 23 of the tube 22 collapses the tube 22, expelling material contained in the tube 22 out of the tube 22 through the opening 23.

For example, in one embodiment, the slot 26a is configured to redimension (or shape) the proximal end 22p of the tube 22, upon collapse of the tube 22. The redimensioning includes processes for changing the dimensions of the portion of the tube 22 that is collapsing, other than the flattening of the tube 22, resulting from the aforementioned collapse and passage through the slot 26a. Redimensioning includes one or more processes performed on the tube 22 upon its collapse, by the pressure member 26, such as gathering tube 22 edges, folding or bending these edges at the proximal end 22p of the tube 22, so that the now flattened proximal end 22p of the tube 22 has a width equal, approximately equal to, or less than, the diameter, or other largest dimension, of the filled (non-collapsed) portion of the tube 22. This allows the tube 22 to remain in conformance the width/diameter of the inner channel 403 of the guide member 400, as detailed below. Alternatively, the slot 26a shown may be linear, as well as other shapes, dimensions and configurations, such as angled, rounded, curved and the like.

The pressure member 26 is made, for example, of metal, plastic, or the like, typically surgical grade, and is, for example, rigid. The pressure member 26 is of sufficient hardness, shape and dimensions, to cause collapsing and flattening of the tube 22, when the pressure member 26 is moved distally (in the direction of the arrow 100), from an initial position, as shown in FIG. 7A, to an advanced position, as shown in FIG. 7B, as material is forced out of the tube 22 through the opening 23. In this preferred but non-limiting example illustrated in FIGS. 7A and 7B, in moving between the initial position of FIG. 7A and the advanced position of FIG. 7B, the pressure member 26 has moved along at least the majority of the initially-filled portion of the tube 22.

The pressure member 26 is shown as suitable for manual advancement (distally), but may be advanced by other mechanical, as well as pneumatic, hydraulic and electrical apparatus or components. The pressure member 26 may be prefitted onto the tube 22, at the proximal end 22p, or placed onto the tube 22 proximate to or at the time of use.

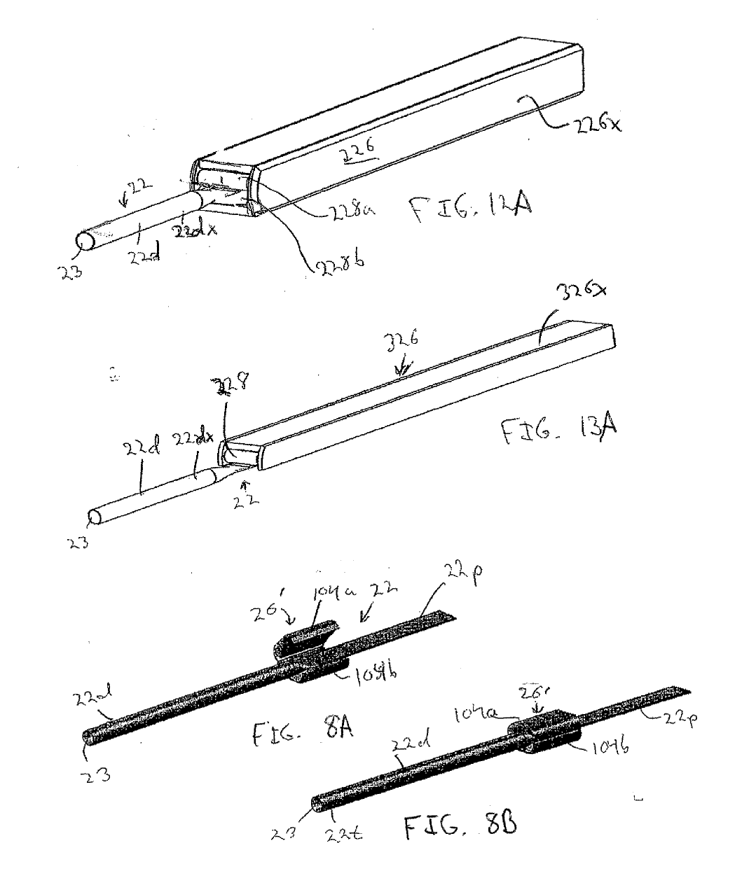

While the pressure member 26 as shown is of a single piece, the pressure member may be made of multiple pieces. For example, as shown in FIG. 8A, the pressure member 26′ is a single piece, formed of sections 104a, 104b hinged together. The pressure member 26′ is clamped over the tube 22 and the sections 104a, 104b, engage each other in a temporary or permanent locking engagement, enveloping the tube 22, as shown in FIG. 8B. The locking engagement is such that the pressure member 26′ remains movable over and along the tube 22.

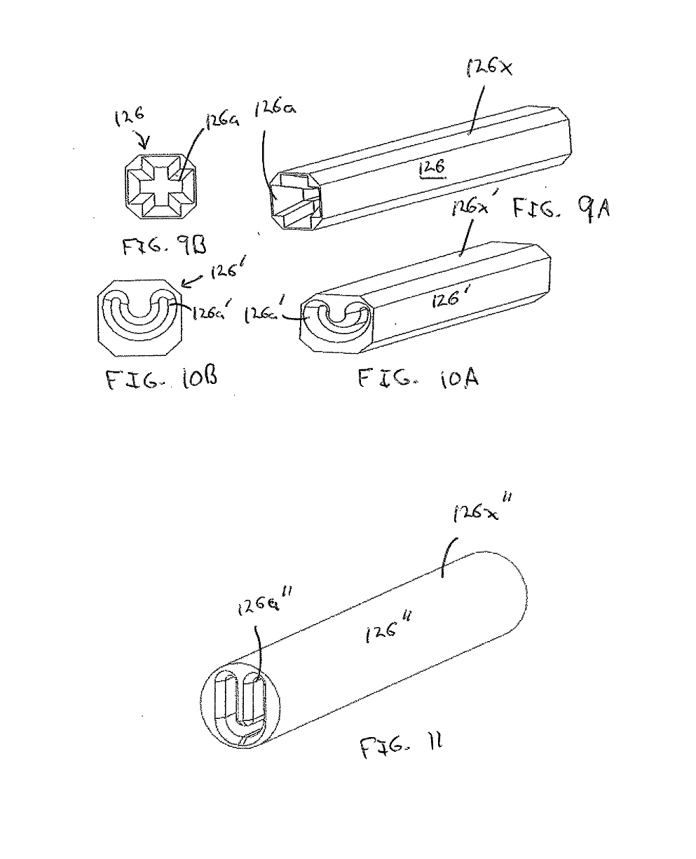

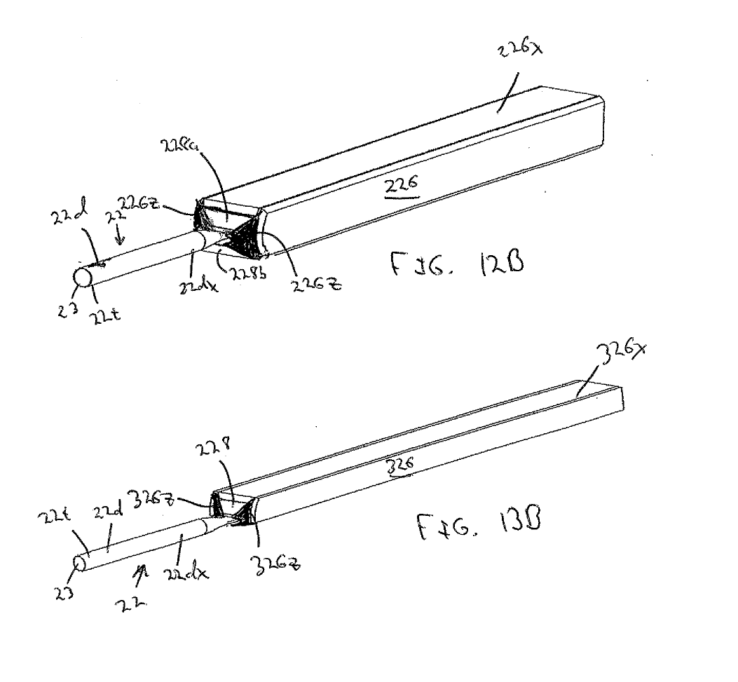

Alternative pressure members include pressure members 126 (FIGS. 9A and 9B), 126′ (FIGS. 10A and 10B), and 126″ (FIG. 11). The pressure members 126, 126′, 126″ include slots 126a (cross shaped in FIGS. 9A and 9B), 126a′ (“C” shaped in FIGS. 10A and 10B), 126a″ (“U” shaped in FIG. 6). These slots 126a, 126a′, 126a″ receive the tube 22, for example, a flattened portion thereof, at the proximal end 22p of the tube 22, and collapse the tube 22 upon moving axially (distally) toward the opening 23 in the distal end 22d of the tube 22, as detailed above.

Other alternative pressure members include a pressure member 226, formed of a block 226x, with dual rollers 228a, 228b, defining a constrictor, for constricting the tube 22 between the rollers 228a, 228b, as shown in FIG. 12A. FIG. 13A shows a “one sided” pressure member 326 including a block 326x and single roller 228 or constrictor, for constricting the tube 22, as detailed above. Alternatively, in FIGS. 12B and 13B, respectively, the blocks 226x and 326x of the respective pressure members 226, 326, for example, include members or arms 226z, 326z, which are, for example, curved, distal to the rollers 228a, 228b, 228, for redimensioning the tube 22 upon its collapse by the respective pressure members 226, 326, the redimensioning in accordance with that detailed above. For example, the redimensioning involves bringing the proximal end 22p of the tube inward at the edges (and may also cause folding), so that the proximal end 22p of the tube 22 is flattened with a width equal to, approximately equal to, or less than, the diameter, or other largest dimension, of the filled (non-collapsed) portion of the tube 22. This allows the tube 22 to remain in conformance the width/diameter of the inner channel 403 of the guide member 400, as detailed below.

FIGS. 12C-1 and 12C-2 show alternative rollers 228a′, 228b′ and 228a″, 228b″, for the pressure members 226 of FIGS. 12A and 12B, as discussed above. The rollers 228a′, 228b′ and 228a″, 228b″ are paired and are of cooperating structure in order to achieve the aforementioned redimensioning of the tube 22 during collapse and flattening. The rollers 228a′, 228b′ of FIG. 12C-1 are conical, while the rollers 228a″, 228b″ of FIG. 12C-2 are curved.

Alternatively, the pressure members 26, 26′, 226 and 326 may be such that their movement on and/or along the tube is limited by the apparatus that moves these pressure members 26, 26′, 226, 326. Additionally, the pressure members 26, 26′, 226, 326 may have an insertion side feature, which enables the tube 22, to gradually deform or bend, leading to the tube 22 collapse, as detailed above, for material to exit the tube 22. Other alternative constructions for pressure members include take up mechanisms that allow for the end of the tube, i.e., the proximal end 22p to be rolled up, to cause the aforementioned collapsing of the tube 22.

FIGS. 14A and 14B, and also turning back to FIG. 1, shows the apparatus 20 in an exemplary operation. The tube 22 of the apparatus is either prefilled with material, such as fusion promoting material (FPM) detailed above, or FPM is inserted into the distal end 22d (or proximal end 22p) of the tube 22. The pressure member 26 is placed onto the tube 22 at the proximal end 22p, if it has not been prearranged on the apparatus 20.

A guide member 400 (also known as a delivery conduit) accesses the surgical site by being inserted into the body and moved to a position proximate to the vertebrae 402a, 402b, for example, into a space or volume between the vertebrae 402a, 402b, by conventional surgical techniques. The guide member 400 is for example, a conduit, tube, partial tube, or the like, which accesses the body via a trocar or the like, by conventional surgical techniques. The tube 22 of the apparatus 20 is inserted into the guide member 400 (into a channel or inner channel 403 of the guide member 400) and moved in the inner channel 403 into the desired position at the surgical site, for example, adjacent to the site, in abutment with the site, into the site, into a predefined space in the surgical site (such as a cut out portion of a vertebrae), or into a space made by a device located in the disc space or volume (between the vertebrae 402a, 402b), one device, for example, is the device disclosed in PCT/IB2011/053143, entitled: Surgical Systems and Methods for Implanting Deflectable Implants, the disclosure of which is incorporated by reference herein.

The user grips the handle 30 of the apparatus 20 (at the arms 34, 35), causing the pressure member 26 to move distally and collapse the tube 22, at the distal end 22d, such that material 405 is emitted from the opening 23 of the tube 22 to the surgical site (or volume associated therewith). The speed of the grip and extent of the grip of the handle 30 results in the delivery speed and the amount of material 405 emitted from the tube 22, to the surgical site.

In an alternative delivery mode, the tube 22 may be advanced into the volume to be filled, and then withdrawn from the volume, while the pressure member 26 remains in a fixed position. The material 405 emitted remains in the within the volume to be filled. In this alternative delivery mode, the emission of material 405 is achieved by relative motion of the tube 22 on the apparatus 20, independent of any moving components.

Additional surgical sites where the above apparatus and all other apparatus disclosed herein, are usable and the same or similar procedures can be performed include, for example, spinal discs, vertebrae, bony structures, volumes partially defined by at least one bony structure, volumes partially defined by two bony structures, locations in proximity to the human spine, the human spine, an inner volume of an orthopedic implant positioned at least partially between two vertebral bodies, and an inner volume of an orthopedic implant positioned at least partially inside a vertebral body.

The present invention is suitable for both human and animal use. Additional uses include filling voids in the body, delivering materials to difficult access areas of the body, filling voids in bones or fractures thereof, and also filling and/or cementing non-unions of bones and tissues such as clavicles, tibias, lower lumbar vertebrae, and the like.

It will be appreciated that the above descriptions are intended only to serve as examples, and that many other embodiments are possible within the scope of the present invention as defined in the appended claims.

What is claimed is:

1. A system for delivering material to a surgical site, comprising:
a collapsible tube including a proximal and a distal end, the collapsible tube for being filled at least partially with material; and,
a pressure member moveably mounted to the proximal end of the collapsible tube, the pressure member configured for moving distally along at least a majority of the collapsible tube, such that the distal movement of the pressure member collapses a portion of the collapsible tube and material is emitted from the distal end of the collapsible tube.

2. The system of claim 1, wherein the pressure member includes a constrictor for progressively collapsing the collapsible tube when the pressure member moves along the collapsible tube.

3. The system of claim 2, wherein the constrictor includes at least one of a slot extending through a block of the pressure member, or at least one roller.

4. The system of claim 1, additionally comprising, means for moving the pressure member distally along the collapsible tube.

5. The system of claim 4, wherein the moving means includes at least one of mechanical means, hydraulic means, pneumatic means or electrical means.

6. The system of claim 1, wherein the collapsible tube includes a shaping member at the distal end configured for causing the bone material to be emitted from the collapsible tube in a predetermined shape.

7. The system of claim 1, wherein the collapsible tube is cylindrical in shape and rounded in cross section.

8. The system of claim 1, wherein the collapsible tube is cylindrical in shape and rounded in cross section in a first portion and rectangular in cross section in a second portion.

9. The system of claim 1, wherein the collapsible tube includes at least one protrusion at the distal end, the at least one protrusion configured for interlocking with the surgical site.

10. The system of claim 1, additionally comprising material at least partially filling the collapsible tube.

11. The system of claim 10, wherein the material is selected from the group consisting of: bone fill material and bone fusion promoting material.

12. The system of claim 11, wherein the material is a continuous composition in the collapsible tube.

13. The system of claim 11, wherein the material is in multiple pieces in the collapsible tube.

14. The system of claim 1, additionally comprising a guide member including an inner channel for accommodating movement of at least a portion of the collapsible tube.

15. The system of claim 1, wherein the pressure member is additionally configured for redimensioning the portion of the collapsible tube that is collapsing.

16. The system of claim 15, wherein the pressure member includes a slot extending through the pressure member, and configured for redimensioning the tube by gathering the tube from its edges and bending the edges so that the now-flattened portion of the tube has a width less than or equal to the width of the diameter of the non-collapsed portion of tube.

17. A method for delivering material to a surgical site comprising:
providing a material delivery system comprising:
a collapsible tube including a proximal and a distal end, the collapsible tube filled at least partially with material; and,
a pressure member moveably mounted to the proximal end of the collapsible tube, the pressure member configured for moving distally along at least a majority of the collapsible tube, the collapsible tube, such that the distal movement of the pressure member collapses a portion of the collapsible tube and material is emitted from the distal end of the collapsible tube;
accessing the surgical site with the material delivery system, such that the distal end of the collapsible tube is at a location least proximate to the surgical site; and,
delivering material to the surgical site by moving the pressure member distally along the collapsible tube such that the tube collapses and the collapse causes material to be emitted from the collapsible tube at the distal end.

18. The method of claim 17, additionally comprising:
accessing the surgical site with a guide member including an inner channel; and,
advancing the material delivery system through the inner channel of the guide member to the location at least proximate to the surgical site.

19. The method of claim 18, wherein the collapsible tube includes at least one protrusion at the distal end, the at least one protrusion configured for engaging a spinal disc.

20. The method of claim 18, wherein the surgical site includes a spinal disc.

21. The method of claim 18, wherein the surgical site includes a bony structure.

22. The method of claim 18, wherein the surgical site includes a volume partially defined by at least one bony structure.

23. The method of claim 18, wherein the surgical site includes a volume partially defined by two bony structures.

24. The method of claim 18, wherein the surgical site is located in proximity to the human spine.

25. The method of claim 18, wherein the surgical site is at least partially included within a human spine.

26. The method of claim 18, wherein the surgical site includes an inner volume of an orthopedic implant positioned at least partially between two vertebral bodies.

27. The method of claim 18, wherein the surgical site includes an inner volume of an orthopedic implant positioned at least partially inside a vertebral body.

28. The method of claim 18, wherein the surgical site includes a vertebrae, and additionally comprising: making a cavity in the vertebrae, and, delivering the material to the cavity.

29. The method of claim 17, wherein the material is selected from the group consisting of bone fill material and bone fusion promoting material.

30. The method of claim 17, wherein the material is a continuous composition in the collapsible tube.

31. The method of claim 17, wherein the material is in multiple pieces in the collapsible tube.

32. The method of claim 17, additionally comprising: redimensioning the portion of the collapsible tube that is collapsing.

33. The method of claim 32, wherein the redimensioning includes gathering the tube from its edges and bending the edges so that the now-flattened portion of the tube has a width less than or equal to the width of the diameter of the non-collapsed portion of tube.
